(19) United States
(12) Patent Application Publication
(10) Pub. No.: US 2024/0090882 A1
(43) Pub. Date: Mar. 21, 2024

(54) IMPLANTABLE FULLY ENDOVASCULAR MAMMALIAN BODY SUCCEDENT CAVITY WALL BREACH SEALING DEVICE

(71) Applicant: Puzzle Medical Devices Inc., Montreal (CA)

(72) Inventors: Gabriel GEORGES, Quebec (CA); Yves-Antoine CRÊTE, Montreal (CA); François TRUDEAU, Quebec (CA); Philippe GENEREUX, Basking Ridge, NJ (US)

(21) Appl. No.: 17/990,322

(22) Filed: Nov. 18, 2022

Related U.S. Application Data
(63) Continuation of application No. PCT/IB2021/054395, filed on May 20, 2021.
(60) Provisional application No. 63/027,729, filed on May 20, 2020.

(51) Int. Cl. A61B 17/00

(52) U.S. Cl. CPC A61B 17/0057; A61B 2017/00004; A61B 2017/00588; A61B 2017/00592; A61B 2017/00597; A61B 2017/0061; A61B 2017/00623; A61B 2017/00672; A61B 2017/00676; A61B 2017/00884

(57) ABSTRACT

Implantable fully-endovascular mammalian body succedent cavity wall breach sealing device comprising a support frame having compact and expanded configuration. Cavity wall barrier membrane tensioned by the frame. Membrane having a tensioned penetrable zone when. Device having a delivery configuration in which the frame is in its compact configuration, the membrane is unpenetrated, and the device is deliverable transcatheterly to a remote implantation site. Deployed configuration in which the frame is in its expanded configuration for anchoring the device in place at the implantation site. Device being positionable with such that when in its deployed configuration it has a single lumen in continuity with a native fluid flow path within the body cavity formed by the support frame. Penetrable zone abuts wall of the body cavity to be succedently traversed by a catheter. Penetrable zone permitting penetration of the catheter into the single lumen, and self-sealing around an exterior surface thereof.

### CROSS-REFERENCE

The present application is a continuation of International Application No. PCT/IB2021/054395, filed May 20, 2021, which claims priority to and the benefit of U.S. Provisional Patent Application No. 63/027,729, filed May 20, 2020, entitled “Vascular Hemostatic Membrane and Method of Implantation”, the contents of which are incorporated herein by reference in their entirety for all purposes.

### FIELD

The present technology relates to endovascular mammalian body cavity wall breach sealing devices and systems.

### BACKGROUND

In 1953, Sven Ivar Seldinger described a technique for obtaining safe access to human body vessels and hollow organs. Today, this technique is known as the Seldinger technique, and it is commonly used. At a high level, the technique consists of puncturing (breaching) the vessel with a needle to gain access to its lumen, then inserting a guidewire into the vessel via the needle, then removing the needle and railing a dilator assembly over the guidewire. The purpose of the dilator assembly is to enlarge the hole so that it is of a sufficient size to pass a catheter (e.g., including a delivery sheath) of a desired size through it.

The Seldinger technique has become the gold standard to access body cavities (e.g., vessels and hollow organs) in order to be able to perform transcatheter techniques. Today, it is used by interventional radiologists, interventional cardiologists, cardiac surgeons, vascular surgeons and other specialists.

The technique's widespread use has enabled outstanding developments in the field of cardiology, where transcatheter interventions currently include coronary stent placements, endovascular grafts, vascular stent placements, transcatheter aortic valve replacements (TAVR), etc. Today, transcatheter techniques are minimally invasive techniques that have, in many instances, replaced traditional open surgical techniques that require open repair. Transcatheter techniques offer a lower intervention risk profile, shorter intervention and recuperation times, and are usually preferred by patients as they are less invasive. For example, TAVR is now recommended over open surgical aortic valve repair in both high and low risk patients.

Transcatheter techniques include both “percutaneous” techniques and “cutdown” techniques. In “percutaneous” interventions the target vessel is punctured with a needle that passes directly through the patient's skin. In “cutdown” interventions, the target vessel is first surgically exposed before being punctured with a needle. As between the two, cutdowns allow for better control over the vessel in the event of bleeding and for larger diameter catheters or devices to be more easily inserted into the vessel. (E.g., with a surgical cutdown, access site hemostasis may be surgically obtained using purse-string sutures around the catheter). Cutdowns also pre-expose the vessel, which better allows for repairs in case of iatrogenic vascular trauma.

Nonetheless, percutaneous inventions tend to be performed more often as they do not require a surgeon, they do not require an operating theatre to be performed in, they are carried out under local anesthesia, and they are quicker to perform.

One of the drawbacks to percutaneous transcatheter interventions is, however, the lack of direct vessel access allowing for control of bleeding during and after the intervention. For example, bleeding typically occurs when large bore implements (e.g., a catheter, typically over 14 Fr) are removed from a vessel and also when there is an exchange of a larger bore implement for a smaller bore implement (e.g., replacement of a 21 Fr sheath with a 14 Fr catheter). This may occur as inserting a large bore implement (e.g., catheter) stretches the vessel wall at the entry site, which hinders the wall's recoil capacity for when the large bore implement is removed or exchanged for a smaller-sized implement. This is especially the case for elderly patients whose vessels are often calcified.

On another note, bleeding is much more frequent in arterial access procedures (i.e., when the target vessel is an artery; both percutaneous and cut-down procedures), as compared with venous access procedures (i.e., when the target vessel is a vein), because of the higher blood pressures found in arteries. With arterial access procedures, hemostasis cannot always be obtained with manual compression. In extreme situations, if serious arterial bleeding is left undetected, patient death can occur.

As a growing number of large bore percutaneous procedures are being performed, bleeding incidence is increasing. Multiple hemostasis systems have been developed to overcome this problem after the intervention is completed. Such systems include both “pre-closure” devices and “post-closure” devices. Pre-closure devices are typically implanted after obtaining vessel access, but before the insertion of an implement (such as a large bore catheter) through the access opening. Pre-closure devices typically provide for hemostatic blood vessel wall sutures that are implanted in the patient before the large bore implement is inserted. The sutures are present throughout the procedure, and they are finally tightened to obtain hemostasis after the implement (e.g., catheter) has been removed. Two commercially available pre-closure systems are the Perclose™ ProGlide™ system and the Prostar XL™ system, both available from Abbott™.

By contrast, post-closure devices are typically inserted after all of the implements except for the guidewire have been removed from the patient. Typically, post-closure devices operate on the principle of filling the hole left by the large bore access with collagen, patches, membranes, etc. (i.e., “plugging up” the hole). Present or formerly commercially available post-closure systems include the MANTA™ device available from Teleflex™, the PerQseal™ available from Vivasure Medical™ and InSeal™ device developed by InSeal Medical™.

In addition to the aforementioned commercial systems, the patent literature describes a whole host of other systems and devices, both pre-closure and post-closure, designed to provide hemostasis after transcatheter interventions. Some examples include:

International Patent Application Publication WO 2008/027366 A1, published Mar. 6, 2008, entitled “Devices and Methods for Creating and Closing Controlled Openings in Tissue”, which purports to disclose: “Devices and methods for creating and closing controlled, shaped openings in tissue, such as blood vessel walls or fascia layers, utilize an implantable access and closure device, or access port, with an aperture that demarcates a desired location for the opening in the tissue. Incision means for creating an opening through the tissue may include a crescent or arc-shaped heating element; a cutting wire; a water jet; or a cutting edge that is integrated into the access port. A self-sealing incision is also described. Closure means to seal the arteriotomy or other opening and provide hemostasis may include a net, patch, ring or wire that is deployed externally to the vessel, covering the arteriotomy site. Another closure means comprises a stent, or tube-like closure mechanism that is inserted through the arteriotomy site and deployed within the vessel. A side opening allows a procedure sheath to enter the blood vessel. After the procedure, the closure mechanism is rotated within the vessel so that the side opening is no longer aligned with the arteriotomy.” (Abstract.)

United States Patent Application Publication No. 2012/0203328 A1, published Aug. 9, 2012, entitled: “Scaffold Device for Preventing Tissue Trauma”, now U.S. Pat. No. 8,685,947 B2, which purports to disclose: “A scaffold is provided for managing access through tissue. The scaffold can support the tissue during medical procedures. The scaffold is placed around or proximate an opening in tissue. The scaffold can expand when medical devices are introduced through the scaffold and through the opening and retract when the medical devices are removed. When the medical devices are removed, the scaffold closes automatically to substantially close the opening.” (Abstract.)

United States Patent Application Publication No. 2014/0066979 A1, published Mar. 6, 2014, entitled: “Device and Method for Closure of a Body Lumen”, now U.S. Pat. No. 10,912,547 B2, which purports to disclose: “A medical device and method for closure of a puncture in a body lumen are disclosed. The device has an aggregate (10) of a support structure (20) and a substantially fluid tight patch member (30) attached thereto at an attachment unit (40). The aggregate has a first, temporary delivery shape, for delivery to an interior of said body lumen and to be subsequently subjected to a change of shape to a second shape, which is a tubular shape. When delivered in said body lumen, the patch member is arranged radially outside of said tubular support structure and arranged towards an inner tissue wall of the body lumen. The aggregate is the detached from a delivery device and said puncture is intraluminally closed in a leakage tight manner, advantageously supported by a physiological pressure of a body fluid in said body lumen. Rotational orientation is detectable by fiducial markers. A compact delivery configuration is provided by the patch being attached to the support structure at a single radial attachment position only.” (Abstract.)

One thing that all of the aforementioned post-percutaneous-transcatheter-intervention closure systems and devices have in common is that they are all designed with the objective to obtain homeostasis after the intervention has been completed and all of the implements have been retrieved from the vessel lumen. None of these closure systems were developed with the objective to obtain hemostasis at a vessel access site at which an implement is left in place (i.e., exits the patient through the vessel access) for a prolonged period of time (e.g., multiple days, weeks, months, years). Indeed, none are suitable for such a use case. One of the reasons why hemostasis is difficult to obtain when an implement is left in place is that patient movement will translate into movement of the lips of the vessel entry point, which inhibits proper scaring of the vessel wall around the implement. This is particularly the case for degenerated vessels in older patients, where calcified plaques hinder the vessel's natural compliance and elasticity.

This issue is not a theoretical one, as cardiological interventions have been developed and others proposed in which a catheter is left in place after retrieval of a larger bore sheath. For example, endovascular micropumps, such as the Impella™ devices available from Abiomed™ have been developed to unload a patient's heart in the setting of heart failure. Impella devices are currently approved for short-term support of patients with cardiogenic shock, and these devices are sometimes used as a short bridge-to-transplant or bridge-to-decision. An initial delivery sheath of an Impella micropump features a profile as high as 21 Fr (depending on the model), while the catheter left in place (for transferring power from the controller to the endovascular micropump) has a profile of 11 Fr or less (again depending on the model). As the catheter is left in place, the vascular seal is suboptimal and access site bleeding is a major and common complication with Impella device implantation and usage. Suture-based techniques are commonly used to try and avoid bleeding complications in patients undergoing such procedures, but such systems are clearly not optimal.

This issue will continue to present itself as multiple other endovascular pump technologies are currently in development. All of these systems require that a catheter be left in place through the vessel access site when in use. This catheter typically contains electrical wires to power an endovascular motor to drive a pump impeller or a flexible driveshaft to rotate the impeller of the pump. For example, the Procyrion™ Aortix™ device is an intra-aortic analog to the trans-aortic Impella devices that is currently in investigation for high-risk percutaneous coronary intervention (“PCI”). Additional catheter requiring systems are currently in development, such as, for example, the devices developed by Magenta Medical™ by Second Heart Assist™, the recently approved Heartmate PHP™ by Abbott™ and the device described in commonly-owned International Patent Application Publication No. WO 2020/198765 A2, published Oct. 1, 2020, entitled “Modular Mammalian Body Implantable Fluid Flow Influencing Device and Related Methods” (hereinafter referred to as the WO '765 Publication. (This list is exemplary, not exclusive. The contents of the WO '765 Publication are incorporated herein by reference in their entirety for all purposes.)

A second aspect common to all of the aforementioned closure systems and devices have in common is that they are all designed to be single use only. None of these closure systems were developed with the objective of allowing for multiple uses over time (e.g., to reopen the closed opening for a second or subsequent use and then to reclose it again after that use). For example, in a suture-based closure system such as the Perclose ProGlide mentioned above, once the sutures have been tightened and the knots tied, the excess external wires are cut. If access to the patient's vasculature is required after closure, it is not possible to release those sutures. A new access site and closure system will need to be used. For the “plug-based” closure systems, the plug materials were not developed to permit satisfactory hemostasis to be reachieved at the end of a procedure in which the plug materials had been punctured for the purpose of a second-time access to the patient's blood vessel. Said another way, the previously delivered closure system cannot serve to achieve hemostasis a second time.

Furthermore, when trying to access the patient's vasculature at a preferred site for such an intervention (e.g., the middle third of the common femoral artery), the presence of a closure system from a previous intervention would be undesirable. Indeed, the presence of such a system from a previous intervention may actively discourage physicians from accessing the vessel at that same location as the first access. This would likely be the case as the closure system's remnants (e.g., the sutures or plug material) may have negative effects, such as for example: (i) reducing the vessel's elasticity in the vicinity of that location; (ii) disrupting the previously obtained hemostasis by reopening the closed vessel wall at a scarred site; (iii) damaging the remain closure system material, which could then embolize into the bloodstream or protrude into the vessel lumen and form an occlusive thrombus. In view of these risks, a physician may avoid the previous access site altogether and choose a different, suboptimal, puncture site.

On a different note, another crucial situation in which hemostasis must be achieved is when performing an intervention that uses a transcatheter transcaval technique. A transcaval technique involves first obtaining transcatheter venous access to the patient's vena cava via a peripheral vessel such as a femoral vein. Next, both vena cava vascular wall and the aortic wall are punctured to gain access to the aortic lumen from the vena cava (i.e., the catheter will pass from the lumen of the vena cava through both the puncture in the vena cava wall and the puncture in the aortic wall and finally into the lumen of the aorta. Using a transcaval technique allows for the transcatheter delivery of material into the arterial circulation (e.g., a TAVR valve) while not having to pass the catheter through tortuous, calcified or small arterial peripheral vessels. Hemostasis at the vessel wall puncture sites is obtained at the end of the procedure by using a a device such as an Amplatzer™ occluder, which essentially works as a vascular plug or closure device. (An Amplatzer occluder can also be used to close other vascular communications such as patent foramen ovals between the two atria of the heart.) An Amplatzer occluder completely seals native, iatrogenic, or intentional inter-vascular communications. In an instance where a catheter would need to remain in place through such an intercommunication pathway after the end of a procedure, neither an Amplatzer occluder nor any other available closure could be used. Thus, the procedure could not currently be performed, as there would be a serious risk of massive internal bleeding. (An example of such a procedure would be the transcatheter transcaval implantation into the arterial system of a microaxial pump for unloading the heart, as this would require that a catheter extending from the pump to exterior of the patient remain in place in order for the pump to be operated.)

United States Patent Application Publication No. US 20118/0214141 A1, published Aug. 2, 2018, entitled “Systems, Apparatuses, and Methods for Vessel Crossing and Closure” purports to disclose: “An implant includes a collapsible tubular body, which, in an expanded configuration, extends from a first end to a second end centered along a longitudinal axis. The implant includes a hub coupled to the tubular body between the first and second ends. The hub is configured to removably connect to a deployment device. The deployment device is configured to manipulate and position the implant towards an implantation site in a vessel of a patient.” (Abstract). This patent application thus purports to deal with such situations. As far as the present patent applicant is aware however, no commercial product embodying the technology described in that patent application has ever been made available and the applicant for that patent application is no longer operating.

In sum, currently available percutaneous access opening closure systems and devices cannot be used in some situations, and they are not well-suited for use in others. Additional development in this technological area would be desirable.

### SUMMARY

It is thus an object of the present technology to ameliorate at least one of the inconveniences present in the prior art, be it one of those described hereinabove or another. In this respect, although several inconveniences with conventional closure systems for percutaneous transcatheter interventions were described hereinabove, it should be understood that all embodiments of the present technology do not provide relief (i.e., an improved situation) from all of those inconveniences. In some embodiments, the present technology may provide relief from all of those inconveniences. In other embodiments, the present technology may provide relief from some of those inconveniences. In still other embodiments, the present technology may provide relief from only one of those inconveniences. In yet other embodiments, the present technology may provide relief from none of those inconveniences, but relief from one or more inconveniences not specifically described in this document. Finally, in still yet other embodiments, the present technology may provide relief from one or more of the inconveniences described hereinabove and one or more inconveniences not described in this document.

It is a further object of the present technology to provide an improved implantable endovascular mammalian body cavity wall breach sealing device and/or system at least as compared with a prior art device and/or system, be it one of those described hereinabove or another.

The present technology results from a “from scratch approach” to the conception and design of a mammalian body cavity wall breach sealing device. At a very high level, the developer of the present technology has realized that conventional closure systems have a closure device or structure that must be implanted into the body via the opening that one is attempting to “close” (i.e., achieve hemostasis at). Without wishing to be bound by any particular theory, it may be that this is the case as minimally invasive surgical intervention techniques (e.g., transcatheter techniques, endoluminal techniques, endovascular techniques, etc.) were developed long after the development of modern open surgical techniques. With an open surgical technique, once the procedure is completed, the incision is closed from the outside of the body, typically with stitches although more recently with surgical glues in some cases. Making an additional incision is unhelpful in closing existing incisions.

When minimally invasive surgical techniques were developed over the past several decades, “convention wisdom” carried over from open surgical techniques may have led those working in this new art down the same path. I.e., one should not make a second opening into the body to close a first opening one has made into the body. Indeed, one can see evidence of that reasoning in the prior art. (See, e.g., US 2014/00066979 A1, in its background section at para. [0017] disparaging WO 2006/03414 A1 (later published as US 2008/0004652 A1), for this very reason.)

In starting from scratch, the developer of the present technology has attempted to put conventional wisdom aside, and come up with closure devices, systems, and method not bound by this constraint.

The result is that, in one aspect, embodiments of the present technology provide an implantable fully endovascular mammalian body succedent cavity wall breach sealing device. The device comprises a support frame and cavity wall barrier membrane. The support frame has a compact configuration and an expanded configuration. The cavity wall barrier membrane is attached to the support frame and is tensioned by the support frame when the support frame is in its expanded configuration. The barrier membrane has a tensioned penetrable zone when tensioned by the support frame. The device has a delivery configuration and a deployed configuration. In the device's delivery configuration, the support frame is in its compact configuration, the cavity wall barrier membrane is unpenetrated, and the device is deliverable transcatheterly to a remote implantation site within the body. In the device's deployed configuration, the support frame is in its expanded configuration for anchoring the device in place at the implantation site. The device is positionable with respect to the body cavity such that when the device is in its deployed configuration: (i) The device has a single lumen in continuity with a native fluid flow path within the body cavity, the single lumen being formed by the support frame. (ii) The tensioned penetrable zone of the barrier membrane abuts a wall of the body cavity to be succedently traversed by a catheter. The tensioned penetrable zone permits penetration of the catheter through the barrier membrane in the tensioned penetrable zone into the single lumen. The tensioned penetrable zone self-seals around an exterior surface of the catheter when the catheter traverses the wall of the body cavity and penetrates the tensioned penetrable zone of the barrier membrane for reducing outflow of bodily fluid across the wall of the bodily cavity at least while the catheter is in place.

At a high-level the design principle behind a device of the present technology, is, prior to breaching a body cavity (e.g., a body conduit, a hollow organ, etc.) to obtain access to the cavity's lumen (e.g., via the Seldinger technique) to perform a transcatheter intervention, the device is implanted within the cavity with its penetrable zone abutting the wall of the cavity in the area at which it will be breached. In doing so, not only will the wall need to be breached in order to gain access to the lumen, but the penetrable zone will need to be penetrated as well. As the penetrable zone is self-sealing, as wires, catheters, sheaths, etc. are made to penetrate the penetrable zone during the course of the transcatheter intervention, the material and geometry of the barrier membrane within the penetrable zone will self-seal around their exterior, reducing (and depending on the circumstances preventing any) outflow of fluid (e.g., blood) from the cavity during the time that the penetrable zone is penetrated. (Thus, in the context of the present technology “sealing” includes imperfectly sealing (i.e., incompletely sealing), although in many embodiments and implementation perfectly (completely) sealing will be the case.) The device itself is initially implanted through a transcatheter technique, but one in which the size of the access opening (and wall breach) necessary (i.e., the secondary access opening or breach) is smaller compared with size of the access opening (and wall breach) that is necessary to conduct the actual intervention (i.e., the primary access opening or breach).

A device of the present technology is fully endovascular when the entirety of the device when in its deployed configuration is located within the vascular system of the mammalian body. For the purposes of the present disclosure, the vascular system should be understood to include both the blood vessels and the heart. Thus, devices of the present technology are fully endovascular even if they extend within the lumens of different blood vessels and/or extend between the lumen of a blood vessel and a chamber of the heart and/or extend between two different chambers of the heart. Thus, a cavity as used herein includes both the lumens of the blood vessels and the chambers of the heart. A cavity wall includes both the walls of the blood vessels and the walls (septum) and valves of the heart as the case may be. No particular location of implantation within the vascular system is required. Thus, devices of the present technology may be implanted in blood vessel that are directly percutaneously accessible (e.g., a femoral artery) and those that are not directly percutaneously accessible (e.g., the aorta).

As was briefly explained above and will be explained in more detail below, a device of the present technology is implantable within the endovascular cavity prior to that cavity's wall being breached during a transcatheter procedure that will occur later in time (i.e., succedent) to the device's implantation. In this way the device acts to seal a breach in a cavity wall during an intervention (or at least prior to the end of the intervention), as opposed to only at the end of the intervention.

The device has a support frame and cavity wall barrier membrane. The support frame can be any biocompatible structure that is capable of supporting the cavity wall barrier membrane in the manner required by the present technology as described herein. The support frame has a compact configuration and an expanded configuration. In the compact configuration the support frame occupies a reduced volume in order to allow for delivery of the device transcatheterly to the device implantation site. In the expanded configuration the volume occupied by the support frame is larger than in the compact configuration.

The support frame is capable of expanding in vivo at the implantation site from its compact configuration to its expanded configuration. Depending on its design and construction, the support frame may self-expandable or may be required to be expanded by some external element. For example, the support frame may be made of a material that is biased towards the expanded configuration, but which has been constrained by some external element (e.g., a delivery sheath) and will expand once freed from the constraint at the implantation side. In another example, the support frame may be made of a shape memory material (e.g., Nitinol) that is stable at room temperature in its collapsed configuration but converts at body temperature to its expanded configuration (its expanded configuration being the “remembered” shape). In yet another example, the support frame may be made of a biocompatible metal or metal alloy that can be expanded at the implantation site with the balloon being inflated within the support frame (e.g., chromium cobalt).

In some embodiments, the support frame is a wire frame. As the skilled addressee would be aware, wire frames are commonly used in transcatheter cardiological interventions. A common example is a wire stent which is placed in a coronary artery in patients with congestive heart failure. Another example is an anchor used to anchor another device (e.g., a micropump to unload the heart) in place within the vascular system. (See, for example, commonly-owned International Patent Application No. PCT/US2021/012083, filed Jan. 4, 2021, entitled “Mammalian Body Conduit Intralumenal Device and Lumen Wall Anchor Assembly, Components Thereof and Methods of Implantation and Explanation Thereof”; the contents of which are incorporated by reference in their entirety). As a skilled addressee would understand, wire frames of many different designs and constructions are possible.

The cavity wall barrier membrane is attached to the support frame. No particular method of attachment is required. Any method of attachment that is both biocompatible and allows for the barrier membrane to carry out its intended functions in accordance with the present technology may be used. As a non-limiting example, the membrane may be overmolded over the support structure.

The barrier membrane is tensioned by the support frame when the support frame is in its expanded configuration. The barrier membrane has a tensioned penetrable zone when tensioned by the support frame. In the context of the present technology, it is not necessary that the entirety of the barrier membrane be tensioned (although in some embodiments that may be the case). As long as the tensioned penetrable zone is created, and the untensioned portion of the barrier membrane (if one is present in an embodiment) does not interfere with the function of the device, no particular relationship between the tensioned and untensioned portions of the device is required. Similarly, as long as the tensioned penetrable zone is created, it is not required that the entirety of the tensioned portion be penetrable. In some embodiments, impenetrable portions of the barrier membrane that do not otherwise interfere with tensioned penetrable zone may be present. Finally, in some embodiments the barrier membrane may have a single tensioned penetrable zone, and in other embodiments the barrier membrane may have multiple tensioned penetrable zones.

The device has a delivery configuration and a deployed configuration. In the device's delivery configuration, the support frame is in its compact configuration, the cavity wall barrier membrane is unpenetrated, and the device is deliverable transcatheterly to a remote implantation site within the body. In the context of the present technology, no particular distance between the access opening site through which the catheter delivering the device will enter the body and the implantation site for the device is necessary for the two sites to be considered “remote”.

In the device's deployed configuration, the support frame is in its expanded configuration for anchoring the device in place at the implantation site. In many embodiments the support frame will act similarly to a stent or expandable anchor, and in its expanded configuration it will exert sufficient force on the wall of the cavity at the implantation site to anchor the device in place. Thus, in such embodiments, a device with a support frame having particular dimensions in its expanded configuration will be selected by the clinician in order to achieve this anchoring effect. In addition, or alternatively, the device may be anchored in place by projections extending from the support fame and projecting into the wall of the cavity when the support frame is in its expanded configuration.

The device is positionable with respect to the body cavity such that when the device is in its deployed configuration (at the implantation site): (i) The device has a single lumen in continuity with a native fluid flow path within the body cavity, the single lumen being formed by the support frame. In this respect the device does not materially interfere with the native flow of fluid through the cavity. The native flow of fluid continues uninterruptedly through the single lumen formed by the support frame. (ii) The tensioned penetrable zone of the barrier membrane abuts a wall of the body cavity to be succedently traversed by a catheter. During implantation of the device the device is properly aligned with its tensioned penetrable zone being correctly positioned with respect to the area of the cavity wall to be breached later during the intervention (i.e., succedently). Any conventional means of correctly positioning the device may be used. For example, in some embodiments the device has at least one radio-opaque marker to be used with fluoroscopy for assisting in positioning the device.

Once the device is correctly in position and ready for use, the tensioned penetrable zone permits penetration of a catheter through the barrier membrane into the single lumen. This function may be achieved by any suitable structure. For example, in some embodiments the barrier membrane has a single weakened site in the tensioned penetrable zone to permit penetration. In other embodiments the barrier membrane has multiple weakened sites in the tensioned penetrable zone to permit penetration. In either case, depending on the construction and the design of the device, the penetrable zone may (or may not) also be penetrable at locations other than the weakened site(s). A weakened site may be created by any suitable method (or methods). In some non-limiting examples, a weakened site may be a slit through the barrier membrane in the penetrable zone. Alternatively, or in addition, the weakened site may be an almost-complete frangible opening in the barrier membrane that will be easily broken apart and opened by the catheter as it enters. Alternatively, or in addition, the weakened site may be a different material than the material of other areas of the barrier membrane in the tensioned penetrable zone. In some embodiments, the weakened sites are identifiable (for example, via appropriate radio-opaque markers) during the procedure to assist in placement of the catheter.

Weakened sites are not, however, required, and in some embodiments, the tensioned penetrable zone has no weakened sites at all. In some embodiments, the tensioned penetrable zone is penetrable notwithstanding the absence of weaken sites, for example, simply by a clinician puncturing the penetrable zone with a needle or by exerting enough force on the catheter to force it through the material of which the penetrable zone is made.

The tensioned penetrable zone self-seals around an exterior surface of the catheter when the catheter traverses the wall of the body cavity and penetrates the tensioned penetrable zone of the barrier membrane for reducing outflow of bodily fluid across the wall of the bodily cavity at least while the catheter is in place. Depending on the embodiment, the “self-sealing” ability of the penetrable zone is brought into effect in one of any number of different ways. As a non-limiting example, in some embodiments, the barrier membrane material in the tensioned zone is an elastomeric material, e.g., silicone, that will naturally expand as the catheter traverses it but will continue to exert a force around the exterior of the traversing catheter. Thus, there will be little to no gaps between the exterior of the traversing catheter and the barrier membrane material itself, which will reduce or prevent outflow of bodily flood (e.g., blood) from the cavity while the catheter is in place. (As blood coagulates, where the bodily flood is blood, in most instances, the coagulating blood itself would assist in preventing any outflow of blood if the seal made by the barrier membrane is imperfect). Alternatively, or in addition, the device may have elastomeric material positioned around the tensioned penetrable zone (or portions there) to act like an elastic skirt around a penetrating catheter to effect or assist in effecting the reduction of fluid outflow.

Alternatively, or in addition, to barrier membrane material in the tensioned penetrable zone having elastic property, the barrier membrane may have been physically constructed to have structures to provide (or assist in providing) its self-sealing effect. In some non-limiting examples, such structures may include one or more of valves, flaps, hinges, tortuous pathways, etc.

Depending on the embodiment (and thus the design and construction of the device), the tensioning of the penetrable zone of the barrier membrane caused by the support frame adopting its expanded configuration may serve one or more of several different functions that assist in the functioning of the device. For example, the tensioning of the penetrable zone may allow (or more easily allow) the penetration of the catheter through the barrier membrane material in the zone by holding the material taunt, preventing the catheter from simply stretching the material without penetrating it. As another example, the tensioning of the penetrable zone may assist in providing the material with its self-sealing ability by, for example, stretching the material. As another example, the tensioning of the material may provide the material with the appropriate shape to discourage (or prevent) the existence of gaps between the material and the cavity wall which it abuts.

In some embodiments, the penetrable zone further self-seals when the catheter is removed from the barrier membrane. In this manner, in addition to reducing or preventing the outflow of bodily fluid during the time that the catheter is in place penetrating the zone, the material in the zone will also act to reduce or prevent the outflow of bodily fluid after the catheter is removed. Thus, for example, the device may act as post-percutaneous intervention closure device as well.

In some embodiments, the tensioned penetrable zone is one-time penetrable and self-sealing. In other embodiments, the tensioned penetrable zone is repeatedly penetrable and self-sealing. The appropriate device may be selected, for example, based on the procedure to be performed. For example, the procedure to transcatheterly implant a micropump to assist in the unloading of the heart (such as, for example, one of the devices mentioned above) typically requires the insertion of a large bore catheter (delivery sheath) followed by leaving a smaller bore catheter in place (as was mentioned above). Such a procedure ideally requires a tensioned penetrable zone that can be (i) penetrated a first time by a large bore catheter and self-seal around it, (ii) self-seal around a smaller bore catheter when the large bore catheter is removed and dynamically re-self-seal while the smaller bore catheter is in place (e.g., so as to allow the patient to be able to move and not require that they be immobilized), and (iii) self-seal once again, once the smaller bore catheter is removed.

In some embodiments, the tensioned penetrable zone is penetrable by a single catheter at a time. In other embodiments, the tensioned penetrable zone is (i) penetrable by multiple catheters contemporaneously, (ii) self-sealing around each of the multiple penetrating catheters contemporaneously, and (iii) self-sealing as each of the multiple catheters is removed from the barrier membrane. The clinician will select the device appropriate to the intervention to be performed, including taking into account the number of catheters that will need to contemporaneously penetrate the device. Conventionally, it is not possible to obtain hemostasis using a single device when percutaneously inserting more than one catheter into a body cavity, and thus each access opening must be sealed separately.

In some embodiments, the support frame is cylindrical in shape when in its expanded configuration, similar to a conventional cardiac or vascular stent. In some such embodiments, the support frame is dimensioned to fit entirely within a blood vessel of a human when in its expanded configuration. The lumen of the device extends through the cylinder along its longitudinal axis.

In some embodiments the support frame is spherical in shape when in its expanded configuration, forming an open-faced cage ball-like structure. In some such embodiments, the support frame is dimensioned to fit within a chamber of a human heart when in its expanded configuration. The lumen of the device extends through, or may be the entirety of, the interior of the sphere.

In some embodiments, the support frame has a shape of two parallel spaced-apart discs connected together at their central portions by a cylinder, each of the parallel discs and the cylinder has a radius, and the radius of the cylinder being smaller than the radii of each of the discs. In some such embodiments, the tensioned penetrable zone of the barrier membrane extends across the cylinder taken perpendicularly to a longitudinal axis of the cylinder. In some such embodiments, the device may be used in transcatheter interventions simultaneously involving crossing two different body cavity walls with a catheter, e.g., interventions involving a transcaval procedure. Thus, each disc of the device has a single-opening extending through the disc.

In some embodiments, the tensioned penetrable zone of the barrier membrane is a band around a circumference of an outer surface of the support frame when in its expanded configuration. Depending on the design and construction of the device, such a band may facilitate the proper positioning of the device with a body cavity.

In some embodiments, the support frame is a of multi-layered construction and the tensioned penetrable zone of the barrier membrane is disposed at an outer surface of the support frame when in its expanded configuration.

A device of the present technology is not limited to having only a single tensioned penetrable zone in its barrier membrane. In some such embodiments, the tensioned penetrable zone is one of a plurality of tensioned penetrable zones of the device.

In some embodiments, the barrier membrane is resorbable. In this manner, the device may remain implanted within the cavity indefinitely after the procedure and may provide a function, such as to act like a stent, as the case may be. In other embodiments, the support frame is resorbable. In still other embodiments, an entirety of the device is resorbable. In this manner, the device will not remain in the cavity after the procedure for an extended period of time, which may help in preventing long-term complications such as stent thrombosis, inflammatory intimal hyperplasia (thickening of the vessel wall around the foreign body) and infection at the device.

At a high-level, a device of the present technology will typically be used in transcatheter surgical procedures where there is an elevated risk of body fluid, e.g., blood, outflow from a body cavity access opening (e.g., wall breach) used during the procedure. (In this context, “surgical” refers to all procedures where a patient is operated on, including both minimally invasive techniques and open surgical techniques, and not just to open surgical techniques.) As was mentioned hereinabove, non-limiting examples of such transcatheter procedures include those where a low bore catheter through an access opening is used after a high bore catheter had previously been used, where multiple catheters are contemporaneously implanted at the same time through an access opening, where a catheter remains exiting the access opening after the procedure has been completed, etc. The implantation of the device itself precedes the obtaining the access opening to be used (this access opening is generally referred to herein as the “primary access opening”). The implantation of the device itself is a done via a percutaneous procedure, but as this procedure is relatively simple, relatively quick, conventional, and only involves a small-bore delivery sheath, there should be no elevated risk of bodily fluid outflow through the access opening use during the implantation (this access opening is generally referred to herein as the “secondary access opening”). Nor should it be difficult to obtain hemostasis at the secondary access opening after the implantation of the device is completed. Thus, the use of the present technology is not simply a shift of the risk of bodily fluid outflow (e.g., blood leakage) from the primary access opening to the secondary access opening.

Therefore, in another aspect, implementations of the present technology provide a method of reducing or preventing fluid loss (i.e., achieving hemostasis) from a conduit system of a mammalian body through a primary percutaneous access opening into a conduit of the system for performance of a transcatheter surgical procedure, the method comprising:
Prior to obtaining the primary percutaneous access opening (e.g., via the Seldinger technique), surgically obtaining a secondary percutaneous access opening (e.g., via the Seldinger technique) to the conduit system of the body at a site remote from a site for the primary percutaneous access opening. For example, if the site of the primary percutaneous access opening will be in the patient's groin to access the patient's femoral artery, the secondary percutaneous access opening may be into, for example, the contralateral femoral artery, a radial artery or ipsilaterally several centimeters away on the patient's leg to access the same femoral artery.
Guiding a delivery sheath containing a device as described hereinabove in its delivery configuration through the secondary percutaneous access opening to an implantation site within the conduit of the system, the implantation site being at a future entry of the primary percutaneous access opening. As a skilled addressee would understand, this action is carried out using conventional techniques.
Promoting exit of the device from the delivery sheath at the implantation site. Positioning the device such that when the device is in its deployed configuration the tensioned penetrable zone of the barrier membrane of the device abuts the wall of the conduit at the future entry of the primary percutaneous access opening into the conduit. Causing the device to adopt its deployed configuration. As a skilled addressee would understand these actions are carried out using conventional techniques. The actual techniques used will determine the order that the actions are carried out as well as which actions will be simultaneously carried out (if any).
Withdrawing the delivery sheath from the conduit system of the body. As a skilled addressee would understand this action is carried out using conventional techniques.
Closing the second percutaneous access opening into the conduit system. As a skilled addressee would understand this action is carried out using conventional techniques.
Obtaining the primary percutaneous access opening into the conduit system (e.g., via the Seldinger technique) including via penetration of the tensioned penetrable zone of the barrier membrane.

In other implementations, the method comprises:
Performing the method described hereinabove in para. [0057].
Causing a catheter to penetrate through the primary percutaneous access opening (as a skilled addressee would understand this action is carried out using conventional techniques) including the tensioned penetrable zone of the barrier membrane.
Allowing the barrier membrane to self-seal around an exterior surface of the catheter. In the context of the present technology, “allowing the barrier membrane to self-seal . . . ” should be understood as taking no action that would hinder the barrier membrane from self-sealing.

In other implementations, the method of para. [0058] further comprises:
Removing the catheter from the primary percutaneous accessing opening (as a skilled addressee would understand this action is carried out using conventional techniques), including from penetrating the tensioned penetrable zone of the barrier membrane.
Allowing the barrier membrane to self-seal reducing (or preventing) blood loss across the barrier membrane. Again, in the context of the present technology, “allowing the barrier membrane to self-seal . . . ” should be understood as taking no action that would hinder the barrier membrane from self-sealing.

In other implementations, the method of para. [0057] or para. [0058] further comprises:
Causing a second catheter to penetrate through the primary percutaneous access opening (as a skilled addressee would understand this action is carried out using conventional techniques) including the tensioned penetrable zone of the barrier membrane; and
allowing the barrier membrane to self-seal around an exterior surface of the second catheter. Again, in the context of the present technology, “allowing the barrier membrane to self-seal . . . ” should be understood as taking no action that would hinder the barrier membrane from self-sealing.

In other implementations, the method further comprises:
Performing the method of para. a first time, wherein the primary percutaneous opening is a first primary percutaneous opening, and the device is a first device.
Causing a first catheter to penetrate through the first primary percutaneous access opening (as a skilled addressee would understand this action is carried out using conventional techniques) including the tensioned penetrable zone of the barrier membrane of the first device.
Allowing the barrier membrane of the first device to self-seal around an exterior surface of the first catheter. Again, in the context of the present technology, “allowing the barrier membrane to self-seal . . . ” should be understood as taking no action that would hinder the barrier membrane from self-sealing.
Performing the method of para. [0057] a second time, wherein the primary percutaneous opening is a second primary percutaneous opening, and the device is a second device.
Causing a second catheter to penetrate through the second primary percutaneous access opening (as a skilled addressee would understand this action is carried out using conventional techniques) including the tensioned penetrable zone of the barrier membrane of the second device.
Allowing the barrier membrane of the second device to self-seal around an exterior surface of the catheter. Again, in the context of the present technology, “allowing the barrier membrane to self-seal . . . ” should be understood as taking no action that would hinder the barrier membrane from self-sealing.

In another aspect, embodiments of the present technology provide an implantable fully endovascular mammalian body succedent cavity wall breach sealing device. The device comprises a support frame and a cavity wall barrier membrane. The support frame has a compact configuration and an expanded configuration, when in the expanded configuration the support frame is one of a sphere, a spheroid, an ellipsoid, and a conoid. The cavity wall barrier membrane is attached to the support frame and tensioned by the support frame when the support frame is in its expanded configuration. The barrier membrane has a tensioned penetrable zone when tensioned by the support frame. The device has a delivery configuration in which the support frame is in its compact configuration and the device is deliverable transcatheterly to a remote implantation site within the body. The device also has a deployed configuration in which the support frame is in its expanded configuration for anchoring the device in place at the implantation site. The device is positionable with respect to the body cavity such that when the device is in its deployed configuration the device allows for fluid flow within the body cavity, and the tensioned penetrable zone of the barrier membrane abuts a wall of the body cavity to be succedently traversed by a catheter. The tensioned penetrable zone (i) permitting penetration of the catheter through the barrier membrane in the tensioned penetrable zone, and (ii) self-sealing around an exterior surface of the catheter when the catheter traverses the wall of the body cavity and penetrates the tensioned penetrable zone of the barrier membrane for reducing outflow of bodily fluid across the wall of the bodily cavity at least while the catheter is in place.

In another aspect, embodiments of the present technology provide an implantable mammalian body succedent vascular cavity wall breach sealing device. The device comprises a support frame and a barrier membrane. The support frame has a compact configuration and an expanded configuration. When in its expanded configuration the support frame has a shape of two generally parallel spaced-apart discs connected together at their central portions by a hollow cylinder. The hollow cylinder forms a lumen between the central portions of the discs. Each of the discs and the cylinder has a radius. The radius of the cylinder is smaller than the radii of each of the discs. The barrier membrane is attached to the support frame and tensioned by the support frame when the support frame is in its expanded configuration. The barrier membrane has a tensioned penetrable zone when tensioned by the support frame. The tensioned penetrable zone extends across the lumen of the cylinder. The device has a delivery configuration in which the support frame is in its compact configuration and the device is deliverable transcatheterly to a remote implantation site within the body. The device also has a deployed configuration in which the support frame is in its expanded configuration for anchoring the device in place at the implantation site. The device is positionable such that when the device is in its deployed configuration, the tensioned penetrable zone permits penetration of the catheter through the barrier membrane in the tensioned penetrable zone, and the tensioned penetrable zone self-seals around an exterior surface of the catheter when the catheter penetrates the tensioned penetrable zone of the barrier membrane for reducing flow of bodily fluid around the exterior of the catheter.

In some embodiments, when the device is in the deployed configuration at the implantation site, the barrier membrane is positioned on the support frame to reduce outflow of bodily fluid from a cavity wall breach (or two cavity wall breaches) at the implantation site.

### General

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that the use of the terms “first unit” and “third unit” is not intended to imply any particular type, hierarchy or ranking (for example) of/between the units. Nor is their use (by itself) intended imply that any “second unit” must necessarily exist in any given situation.

In the context of the present specification, the word “embodiment(s)” is generally used when referring to physical realizations of the present technology and the word “implementations” is generally used when referring to methods that are encompassed within the present technology (which generally involve also physical realizations of the present technology). The use of these different terms is not intended to be limiting of or definitive of the scope of the present technology. These different terms have simply been used to allow the reader to better situate themselves when reading the present lengthy specification.

Embodiments and implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of embodiments and/or implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description, which is to be used in conjunction with the accompanying drawings, where:

FIG. 1 is an isometric schematic view of a device being a first embodiment of the present technology.

FIG. 2 is a front elevation schematic view of the device of FIG. 1.

FIG. 3 is a side elevation schematic view of the device of FIG. 1 shown having been implanted into a body conduit.

FIG. 4 is a side elevation schematic view of the device of FIG. 1 implanted into a body conduit, during a transcatheter intervention wherein a catheter is shown penetrating the penetrable zone of the barrier membrane of the device, entering and continuing within the body conduit.

FIG. 5 is a side elevation schematic view similar to FIG. 4, shown later in time when the wire frame of the device has been resorbed.

FIG. 6 is a side elevation schematic view similar to FIG. 4, shown later in time when the entire device has been resorbed.

FIG. 7 is a side elevation schematic view similar to FIG. 3, showing a first device having been implanted within a first body conduit and a second device having been implanted within a second body conduit adjacent the first body conduit.

FIG. 8 is a side elevation schematic view similar to FIG. 7, shown later in time with a catheter in use during a transcaval technique.

FIG. 9 is a side elevation schematic view similar to FIG. 4, wherein two catheters are shown penetrating the penetrable zone of the barrier membrane of a device being a second embodiment of the present technology, entering and continuing with the body conduit.

FIG. 10 is an isometric schematic view of a device being a third embodiment of the present technology.

FIG. 11 is a side elevation schematic view of the device of FIG. 10, shown later in time when the device had been implanted into a body conduit during a transcatheter intervention, wherein a catheter is shown penetrating the penetrable zone of the barrier membrane of the device, entering and continuing within the body conduit, when the wire frame of the device has been resorbed.

FIG. 12 is a side elevation schematic view of a device being a fourth embodiment of the present technology.

FIG. 13 is a side elevation schematic view of the device of FIG. 12, shown with a catheter penetrating the two tensioned penetrable zones of the device.

FIG. 14 is an isometric view schematic of a device being a fifth embodiment of the present invention, shown with a catheter penetrating the penetrable zone of the device.

FIG. 15 is a front schematic view of the device of FIG. 14, with the tensioned penetrable zone showing a weakened zone.

FIG. 16 is a front schematic view of the device of FIG. 14, with the tensioned penetrable zone illustrating its self-sealing feature.

### DETAILED DESCRIPTION OF SOME EMBODIMENTS AND IMPLEMENTATIONS

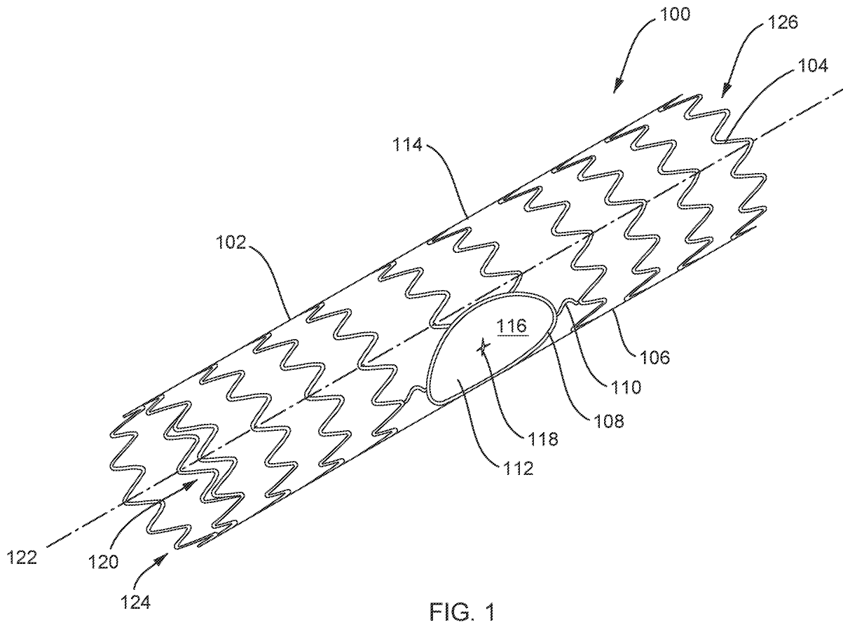

Referring to FIGS. 1 to 3, there is shown an implantable fully endovascular mammalian body succedent cavity wall breach sealing 100, which is one embodiment of the present technology. It is to be expressly understood that the device 100 is merely one embodiment, amongst many, of the present technology. Other embodiments are also described hereinbelow. Thus, the description thereof that follows is intended to be only a description of illustrative examples of the present technology. This description is not intended to define the scope or set forth the bounds of the present technology. In some cases, what are believed to be helpful examples of modifications to the device 100 and/or other embodiments may also be set forth hereinbelow. This is done merely as an aid to understanding, and, again, not to define the scope or set forth the bounds of the present technology. These modifications are not an exhaustive list, and, as a skilled addressee would understand, other modifications are likely possible. Further, where this has not been done (i.e., where no examples of modifications have been set forth), it should not be interpreted that no modifications are possible and/or that what is described is the sole manner of implementing that element or feature of the present technology. As a skilled addressee would understand, this is likely not the case. In addition, it is to be understood that the device 100 may provide in certain instances a simple embodiment of the present technology, and that where such is the case it has been presented in this manner as an aid to understanding. As a skilled addressee would understand, various embodiments of the present technology are of a greater complexity.

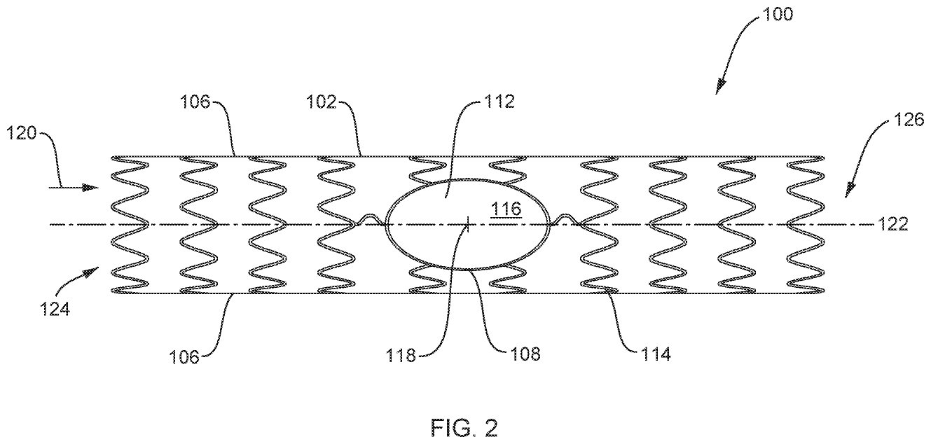

Referring to FIGS. 1, 2 and 3, device 100, being a first embodiment of a device of the present technology, is shown in its deployed configuration. The device 100 has a support frame 102 and a cavity wall barrier membrane 112. In this embodiment the support frame 102 of the device 100 is a wire frame 114. The wire frame 114 is similar to a stent that may be implanted into a patient suffering from peripheral vascular disease. The wire frame 114 has an expanded configuration (in which it is shown in FIGS. 1 and 2) and a collapsed configuration. As can be seen in FIGS. 1 and 2, the wire frame 114, when in its expanded configuration, forms a hollow cylinder (in periphery). The cylinder formed by the wire frame 114 has a central longitudinal axis 122.

The wire frame 114 is composed of a number of wires 104 that form the circumference of the cylinder. As can be seen in FIGS. 1 and 2, the wires 104 are “wavy” in a direction parallel to the longitudinal axis 122 of the cylinder as they travel around the circumference of the cylinder. In this embodiment, this “waviness” increases the effective surface area that each wire will have in contact with the wall of a body cavity when the device 100 is implanted. This “waviness” also ensures that the wire network 114 may be properly collapsed when in its compact configuration. The wires 104 are connected together by a number of straight wires 102 running along the circumference of the cylinder parallel to the cylinder's longitudinal axis 122.

Centrally located between the ends 124, 126 of the wire frame 114, is a wire “circle” 108. The wire “circle” is not actually a circle since it curves around the circumference of the cylinder similarly to the wires 104. The “circle” has a three-dimensional shape of a circle draped over the circumference of a right circular cylinder, but for the purposes of the present disclosure the wire circle 108 is referred to as just that. Because of the three-dimensional shape of the wire circle 108 in the views shown in FIGS. 1 and 2, the wire circle 108 appears as an oval in shape. As can be seen in FIGS. 1 and 2, in this embodiment the wire circle 108 is connected directly to, and interrupts, two of the wires 104 as they travel around the circumference of the cylinder. Further, the wire circle 108 is connected to two other ones of the wires 104 by small curved wires 110.

In this embodiment, all of the wires 104, 106, 108, 110 of the wire frame 114 are made of nitinol. Each of the wires 104, 106, 108, 110 has a diameter of approximately 0.5 mm. (In other embodiments this number will vary.) The cylinder formed by the wire frame 114 (in its expanded configuration) is approximately 50 mm in length and has a diameter of approximately 7 mm. (In other embodiments these numbers will vary.) The “radius” of the wire circle 108 (when the wire frame 114 in its expanded configuration) is approximately 4 mm. The spacing between any two adjacent ones of the “wavy” wires 104 of the wire frame 114 (in its expanded configuration) is approximately 8 mm. (In other embodiments, this number will vary.) It is not necessary that the spacing between each pair of adjacent wires 104 be identical, although in some embodiments that may be the case.

In this embodiment, as can be seen in FIGS. 1 to 3, the cavity wall barrier membrane 112 is associated with the wire circle 108 of the wire frame 114. In this embodiment, the cavity wall barrier membrane 112 is made entirely of a layer of silicone (an elastomeric material). The layer of silicone is 1 mm thick and has a Shore A Durometer of less than 90. (These numbers will vary in other embodiments). The silicone has been overmolded over the wire circle 108 in order to attach the silicone to the wire frame. In FIGS. 1 to 3, as the wire frame 114 is in its expanded configuration, the silicone layer is under tension imparted by the wire circle 108. In this embodiment, again as can be seen in the FIGS., the entirety of the cavity wall barrier membrane 112 lies within the wire circle 108 and is tensioned. Thus, the entirety of the cavity wall membrane 112 in this embodiment is the tensioned penetrable zone 116, and the entirety of the tensioned penetrable zone 116 lies within the wire circle 108 as well. (In other embodiments this will not be the case).

In the “center” of the tensioned penetrable zone 116, the silicone layer has a weakened area 118 in the form of two slits through the silicone layer in a cross formation. In this embodiment, a catheter 60 traversing the tensioned penetrable zone 116 of the cavity wall barrier membrane 112 will generally do so through the weakened area 118. However, other areas of the tensioned penetrable zone 116 may be traversed by a catheter as well in this embodiment.

In addition to its deployed configuration, the device 100 also has a delivery configuration in which it is deliverable transcatheterly to a remote implantation site within a hollow cavity of the patient's body. When the device 100 is in its delivery configuration, the volume occupied by the device 100 as well as its cross-sectional area (taken perpendicular to its longitudinal axis) are greatly reduced to allow for delivery and implantation via a small-bore catheter. Thus, when the device 100 is in its delivery configuration, the wire frame 114 is in its compact configuration in which the wire frame 114 has been collapsed in on itself. Further the silicone layer forming the cavity wall barrier membrane 112 and its tensioned penetrable zone 116, are no longer under tension. Owing to its elastomeric nature, the volume occupied by the silicone layer is itself reduced as the silicone layer retracts.

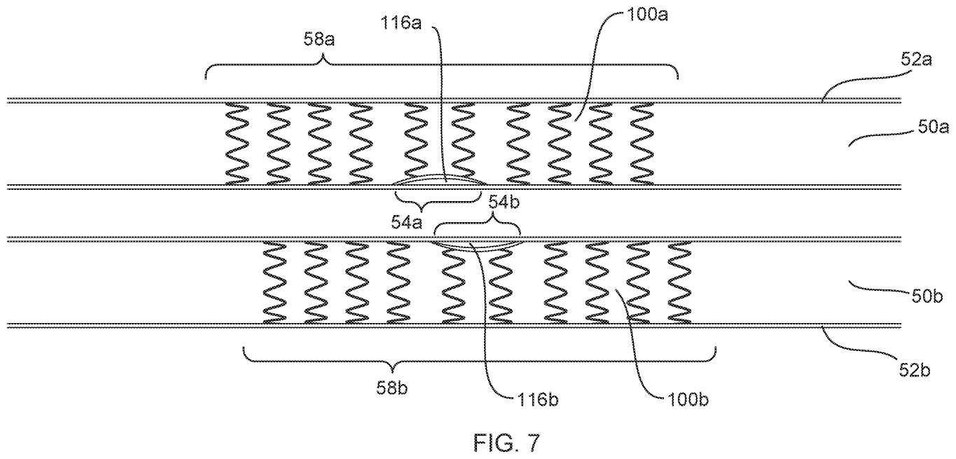

In its compact configuration, the device 100 looks like a small pill shaped structure, see for example, FIGS. 7B, 7C, and 7D, of United States Patent Application Publication No. 2012/0226309 A1, published Sep. 6, 2012, entitled “Device, Kit and Method for Closure of a Body Lumen Puncture” (The entirety of the contents of the US '309 Publication are incorporated herein by reference for all purposes.)

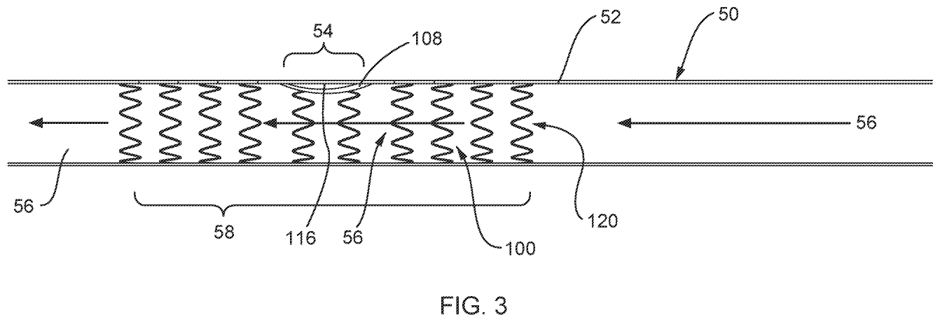

Referring to FIG. 3, the device 100 is shown schematically in a blood vessel 50 in its deployed configuration. In the deployed configuration, the wire frame 114 forms a lumen 120. In this embodiment, the lumen 120 extends from one end 124 to the other end 126 of the wire frame 114 (i.e., through the center of the cylinder parallel to the longitudinal axis 122). During the transcatheter implantation procedure of the device 100, the device 100 is positioned such that the lumen 120 is in continuity with a native fluid flow path within the body cavity. In FIG. 3, the body cavity is a blood vessel 50 and the native blood flow path through the blood vessel 50 is indicated by arrows 56. Thus, as can be seen in FIG. 3, the native blood flow 56 continues through the lumen 120 of the device 100 without any material obstruction.

Further, the wire frame 114 is in expanded configuration anchoring the device 100 in place at the implantation site 58 by exerting a force on the wall 52 of the blood vessel 50. The tensioned penetrable zone 116 of the cavity wall barrier membrane 112 abuts the wall 52 of the blood vessel 50 at an area 54 at which the wall breach made later during the intervention will occur.

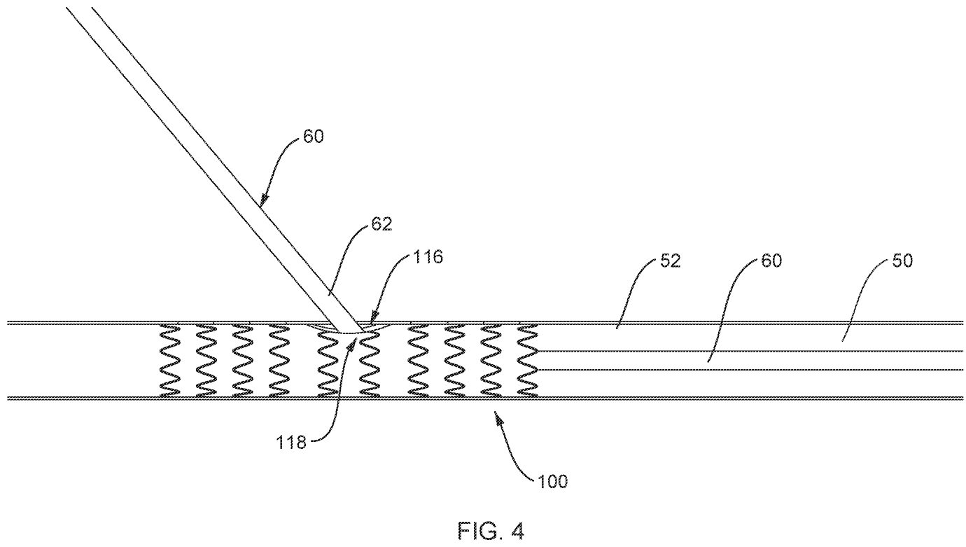

Referring to FIG. 4, the device 100 is shown schematically in a blood vessel 50 in its deployed configuration similar to FIG. 3. However, in FIG. 4, illustrates a later point in time during the transcatheter procedure being performed, at which time a catheter 60 has traversed the wall 52 of the blood vessel 50 (as is conventionally the case with a percutaneous transcatheter procedure), and has penetrated the tensioned penetrable zone 116 of the cavity wall barrier membrane 112 and extends within the blood vessel 50. As the catheter passes through the weakened area 118, the tensioned penetrable zone 116, being made of elastomeric silicone accommodates and self-seals around the exterior 62 of the catheter 60. The barrier membrane 112 thus prevents blood from flowing out of the blood vessel 50 thorough the breach in the wall 52 of the blood vessel 50.

Should the patient move during the time that the catheter 60 extends through the tensioned penetrable zone 116, owing to the elastomeric nature of silicone layer, either the seal around the exterior surface 62 will be maintained or will quickly be re-established if necessary.

In this embodiment, should the catheter 60 be withdrawn from the blood vessel 50 and traversing the penetrable zone 116, the silicone (again owing to it elastomeric nature) will act to close the opening through which catheter 60 traversed. This will reseal the barrier membrane 112 and again act to prevent blood from flowing out of the blood vessel 50 through the breach of the blood vessel wall 52. In this embodiment, the tensioned penetrable zone 116 of the cavity wall barrier membrane 112 is penetrable multiple times. Thus, should it be necessary to do so, a second catheter can be caused to penetrate the penetrable zone 116 and enter the lumen of the blood vessel 50, and the penetrable zone 116 will again self-seal around the exterior of that second catheter, even should bore of that catheter be smaller than that of the first catheter. This process may be repeated as necessary.

When the last catheter 60 is finally removed from the blood vessel 50 and from traversing the penetrable zone 116, the silicone of the barrier layer 112 self-seals and prevents (or assists in preventing) blood from flowing out of the conduit. Hemostasis at the primary percutaneous access opening (through which the catheter 60 had entered the patient) is thus likely to be established much more readily than had the device 100 not been present.

Thus, the use of the device 100 in a transcatheter procedure is as follows:
The primary percutaneous access opening is the access opening through which the transcatheter procedure will be performed. Prior to obtaining the primary percutaneous access opening, surgically obtaining a secondary percutaneous access opening into a blood vessel of the patient at a site remote from the site for the primary percutaneous access opening. (“Surgically obtaining” in this context is not limited to open surgical techniques, it includes minimally invasive techniques; e.g., the Seldinger technique.)
Guiding a delivery sheath containing the device 100 in its delivery configuration through the secondary percutaneous access opening to an implantation site 58 within the blood vessel 50 of the patient, the implantation site 58 being at the future entry area 54 of the primary percutaneous access opening.
Positioning the device 100 relative to the future entry area 54 such that when the device 100 is in its deployed configuration the tensioned penetrable zone 116 of the barrier membrane 112 of the device 100 abuts the wall 52 of blood vessel 50 at the future entry area 54 of the primary percutaneous access opening into the blood vessel 50. Then, promoting exit of the device 100 from the delivery sheath at the implantation site 58 in the proper positioning, which, in this embodiment/implementation causes the device 100 to adopt its deployed configuration.
Withdrawing the delivery sheath from the patient's blood vessel 50, and obtaining hemostasis at the site of second percutaneous access opening; and Performing the transcatheter procedure, including obtaining the primary percutaneous access opening to into the blood vessel 50 including via penetration of the tensioned penetrable zone 116 of the barrier membrane 112.
As was described above, at the end of the transcatheter procedure, hemostasis at the primary percutaneous access opening is likely to be established much more readily than at the device 100 not been present.

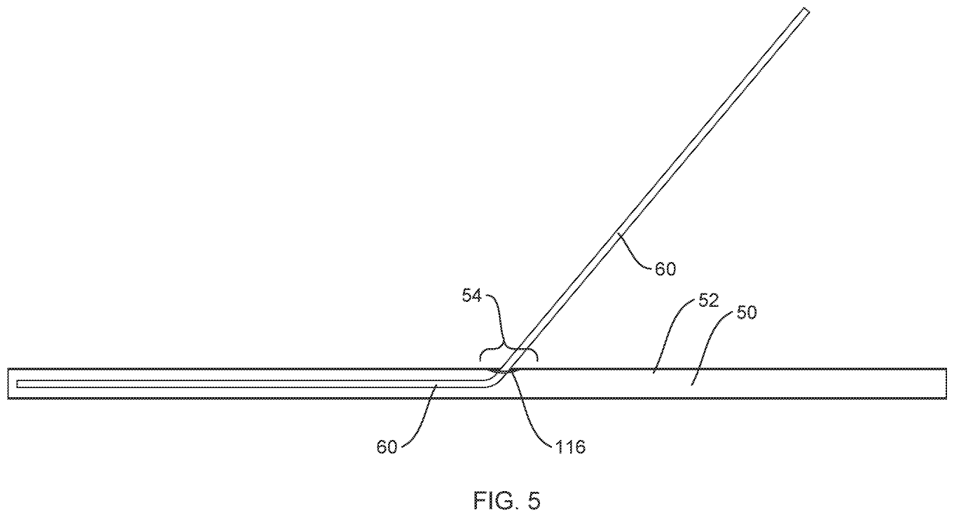

FIG. 5 shows a schematic of an alternate implementation of the present technology, wherein a catheter 60 exits the body of the patient for a relatively long period time. In this implementation, the wire frame of device 100 was made of a resorbable material whereas the cavity wall barrier membrane 116 was not made of a resorbable material. After a period of time, as is shown in FIG. 5, the wire frame of the device 100 will have been resorbed, whereas the barrier membrane 116 remains in place at the site 54 of the blood vessel 50 wall breach (by tissue overgrowth). The catheter 60 remains in place as well.

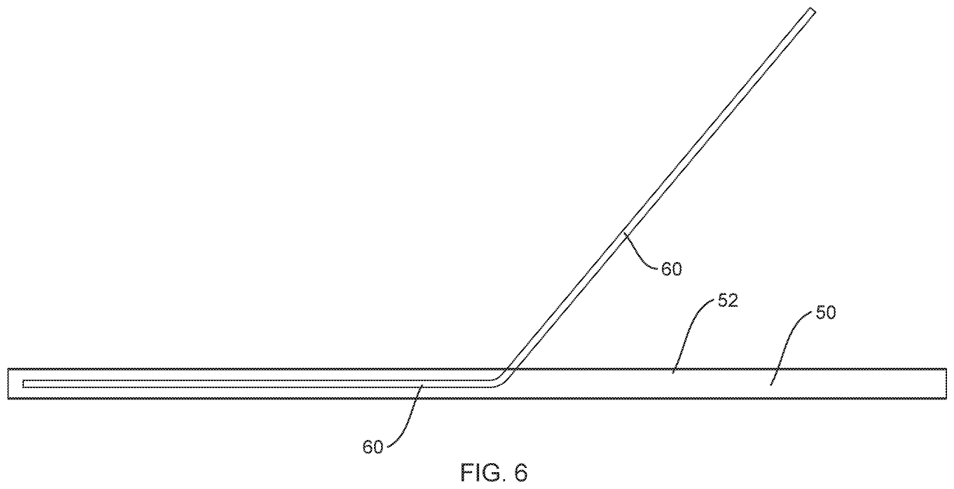

FIG. 6 shows a schematic of another alternate implementation of the present technology, wherein a catheter 60 exits the body of the patient for a relatively long period time. In this implementation, both the wire frame of device and the cavity wall barrier membrane of the device were made of resorbable materials. After a period of time, as is shown in FIG. 6, the entire device 100 will have been resorbed, leaving the catheter 60 in place.

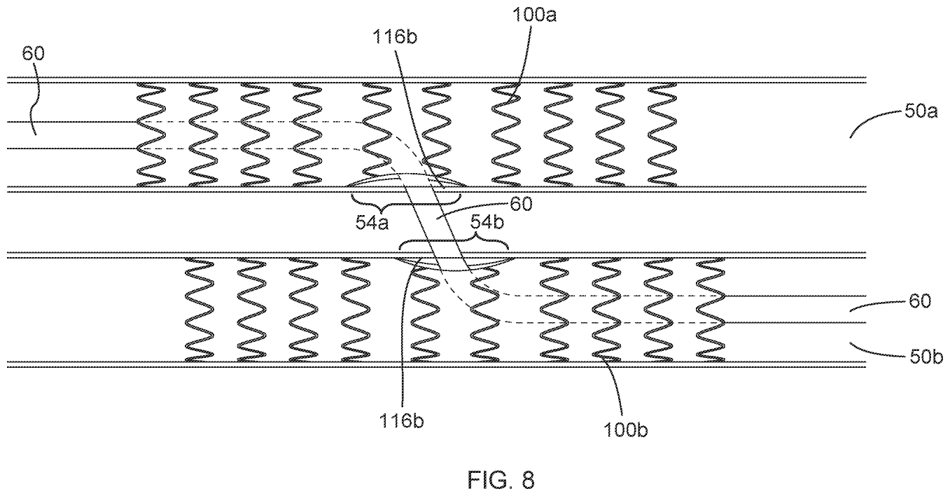

Referring to FIGS. 7 and 8, there is shown schematically another implementation of the device 100 of the present technology, used in what is known in the art as a transcaval technique. (Again, at a high level a transcaval technique is where transcatheter access to the patient's aorta is obtained via the patient's vena cava. Thus, for example, the primary percutaneous access opening for the catheter may be in their groin area into the femoral vein. From that point the catheter travels into the patient's inferior vena cava, then across the walls of the inferior vena cava and of the aorta, then into the aorta and then to its destination site.)

In this implementation, two devices 100a and 100b are used, device 100a in the vena cava 50a and device 100b in the aorta 50b. (There may optionally be a third device, e.g., at the primary percutaneous access opening). Prior to the transcatheter procedure being conducted, each of the devices 100a and 100b are themselves separately transcatheterly implanted in the appropriate locations and appropriately positioned as is required. The primary transcatheter procedure can then be carried out, in which (in addition to proceeding as it conventionally would have) a catheter 60 (FIG. 8) will penetrate the tensioned penetrable zone 116a of the barrier membrane 112a of the device 100a in the vena cava 50a and then will penetrate the tensioned penetrable zone 116b of the barrier membrane 112b in the aorta 50b. In so doing, blood outflow from both the vena cava 50a and the aorta 50b will be reduced, minimized, or prevented (as the case may be).

As the remainder of the details regarding the implantation, use, and explanation of the devices 100a, 100b is the same at that described above with respect to the device 100, those details are not repeated here for the sake of brevity.

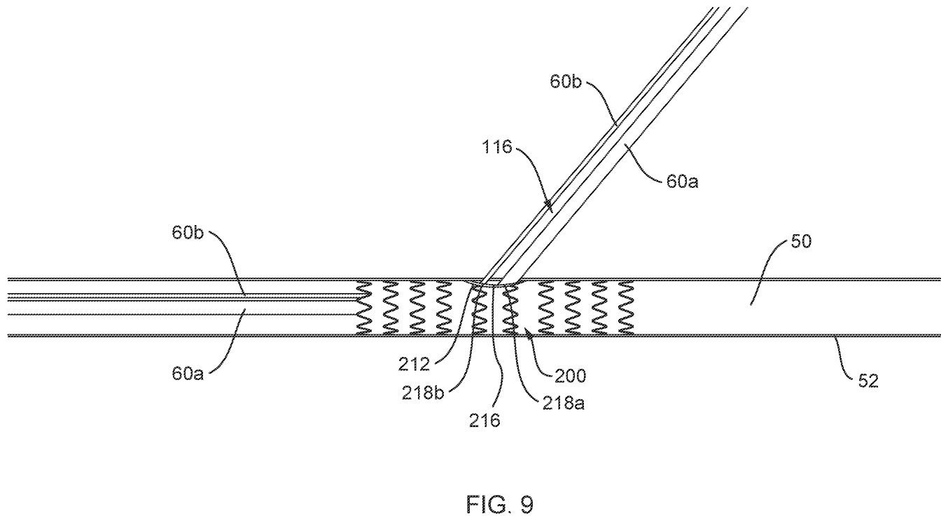

Referring to FIG. 9 there is shown another embodiment of the present technology, device 200. Device 200 is similar to device 100 with the exception described below. Therefore, for the sake of brevity, only that exception is described here. For the remainder of the details, the reader is referred to the description of device 100 above, substituting the reference numbers 2xx for the reference numbers lxx in that description.

In the embodiment shown in FIG. 9, the tensioned penetrable zone 216 of the barrier membrane 212 of the device 200 has two weakened sites 218a, 218b instead of one (as was the case with device 100). Shown in FIG. 9 are two catheters 60a, 60b contemporaneously (at the same time) penetrating the penetrable zone 216, one catheter 60a through first weakened site 218a and a second catheter 60b through second weakened site 218b. The silicone layer of penetrable zone 216 self-seals around the exterior of each catheter 60a, 60b, independently of its self-sealing around the exterior of the other catheter 60b, 60a (respectively). Similarly, silicone layer of the penetrable zone 216 self-seals upon removal of each catheter 60a, 60b independently of its self-sealing upon removal of the other catheter 60b, 60a (respectively). Thus, in this embodiment the clinician may implant, use, and explant each of the catheters 60a, 60b separately as needed during the primary transcatheter procedure without the need to obtain two different primary access openings, one for each catheter 60a, 60b.

Referring to FIG. 10 there is shown another embodiment of the present technology, device 300. Device 300 is similar to device 100 with the exceptions described below. Therefore, for the sake of brevity, only those exceptions are described here. For the remainder of the details, the reader is referred to the description of device 100 above, substituting the reference numbers 3xx for the reference numbers lxx in that description.

In the embodiment shown in FIG. 10, the cavity wall barrier membrane 316 of the device 300 is not a circle wrapped around the surface of a right circular cylinder as was the case with the device 100. In this embodiment, the barrier membrane 316 forms a band around the exterior surface of the device 300 in the center of the device 300 generally equidistant from either of the ends 324, 326 of the wire frame 314. Further, in this embodiment, the wire frame 314 does not have a wire circle, as none is needed. As can be seen in FIG. 10, the barrier membrane 316 is attached to the wire frame 314 at two adjacent “wavy” wires 304 that extend around the circumstance of the wire frame 314 giving it its cylindrical shape. When the wire frame 314 is in its expanded configuration (as is shown in FIG. 3), the entire barrier membrane 316 is tensioned by the wire frame 314 between the two adjacent wavy wires 304. Thus, in this embodiment, as was the case with device 100, the entirety of the barrier membrane 316 is the tensioned penetrable zone 312. In this embodiment, the penetrable zone 316 has no weakened sites, although it is nonetheless still penetrable. In a non-limiting example, device 300 may be advantageously used in situation where it may be difficult to properly position a device, such as device 100, such that its penetrable zone abuts an area of future (succedent) cavity wall breach. As the tensioned penetrable zone 316 of device 310 extends around the circumference, it may, in some situations, be easier to correctly position in its deployed configuration than device 100.

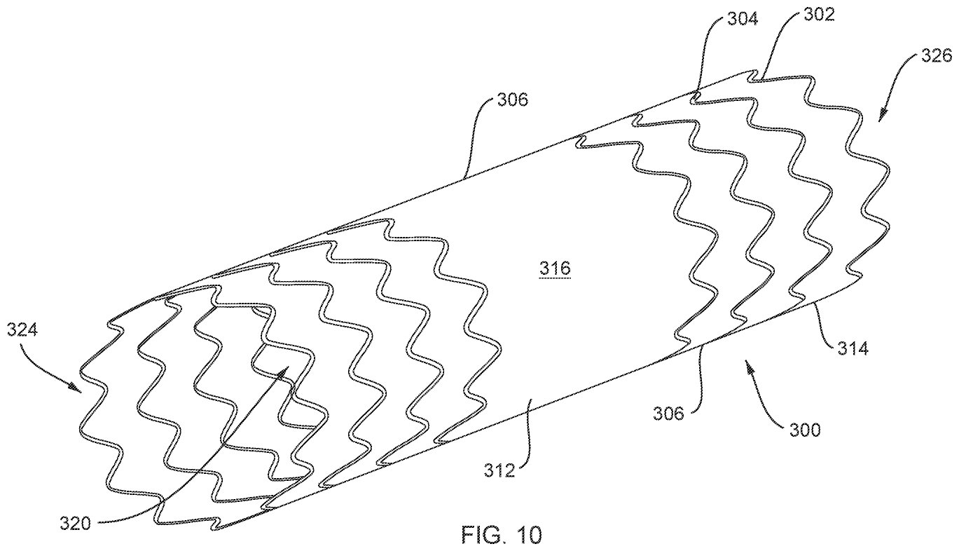

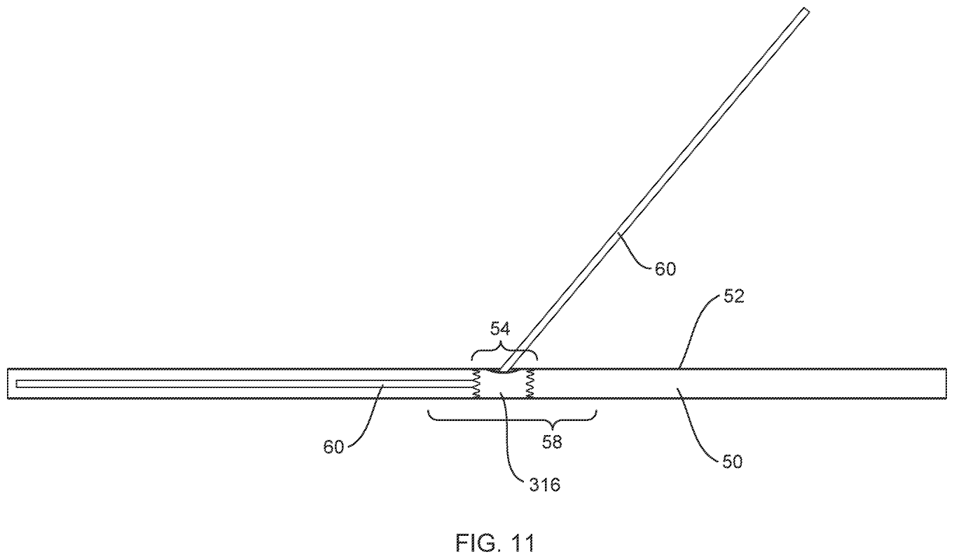

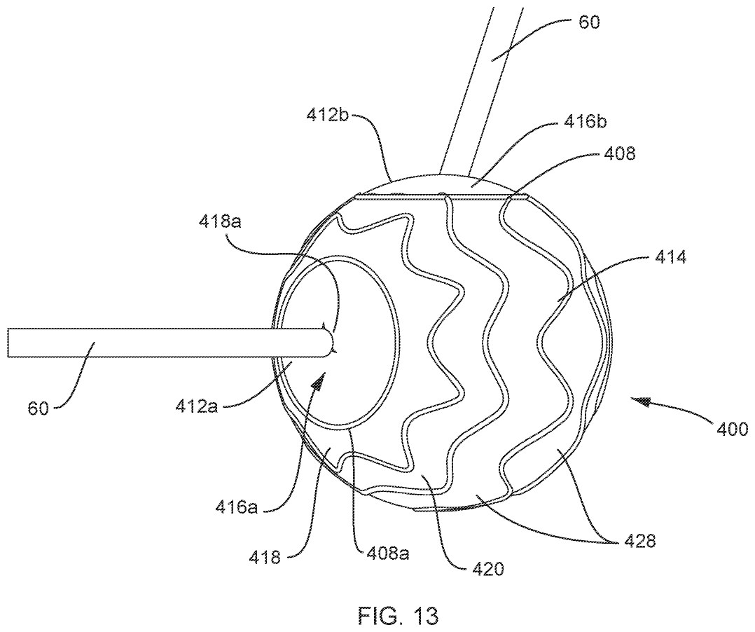

FIG. 11 shows a schematic of an alternate implementation of the present technology, wherein a catheter 60 exits the body of the patient for a relatively long period time. In this implementation, the wire frame of device 300 was made of a resorbable material whereas the cavity wall barrier membrane 316 was not made of a resorbable material. After a period of time, as is shown in FIG. 13, the wire frame of the device 300 will have been resorbed, whereas the barrier membrane 316 remains in place at the implantation site 58 at the site 54 of the blood vessel 50 wall breach (by tissue overgrowth). The catheter 60 remains in place as well.

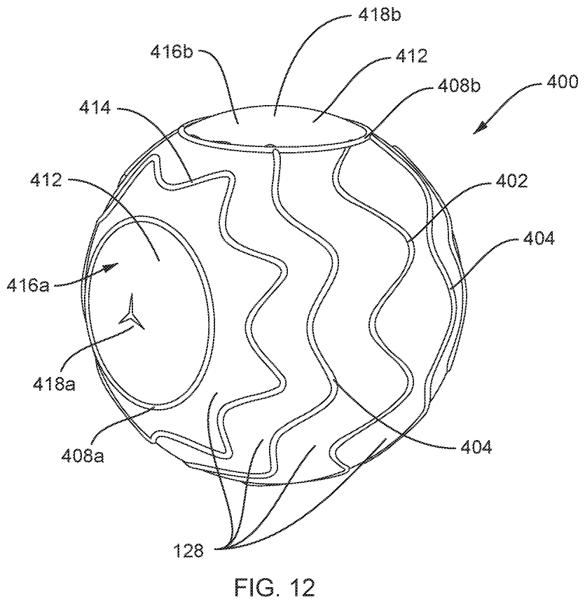

Referring to FIGS. 12 and 13 there is shown schematically another embodiment of the present technology, device 400. In this embodiment, as shown in FIGS. 12 and 13, when the device 400 is in deployed configuration, its support frame 402 (wire frame 414) has the external shape of a sphere. The spherical wire frame 414 is sized to fit within a left atrium of a human adult heart; the left atrium being the body cavity with respect to device 400. The diameter of the spherical wire frame is 40 mm. In other embodiments, the diameter will vary depending on the body cavity in which the device 400 is to be implanted.

In this embodiment, the wire frame 414 is formed by a number of wires 404 that are irregularly shaped and are connected together at various points (only some of which are shown in FIGS. 12 and 13). As can be seen in FIGS. 12 and 13, many of the wires 404 are “wavy” in shape, although the wave forms generally differ between the various wires 404. As was mentioned hereinabove in relation to device 100, the “waviness” of the wires increases the area of the cavity wall over which the wire can exert a force. The “waviness” also ensures that the wire frame 414 can properly compress into its compact configuration. As can also be seen in FIGS. 12 and 13, the wire frame 414 has two wire circles 408a, 408b. In this embodiment, given the difference in the exterior shape of a circle and a cylinder, the wire circles 408a, 408b are actually circular.

In this embodiment, each of the wire circles 408a, 408b serves the same function as does wire circle 108 in the device 100, namely as a support for the cavity wall barrier membrane. In this embodiment, the cavity wall barrier membrane is split into two sections, 412a and 412b. The sections 412a and 412b are not connected together. Each section 412a, 412b forms its own tensioned penetrable zone 416a, 416b (respectively). Each of the tensioned penetrable zones 416a, 416b has its own weakened area 418a, 418b. Each of the weakened areas 418a, 418b is formed of three slits through the silicone layer, the three slits connected at their ends at one common point. Thus, in this embodiment, the entirety of the cavity wall barrier membrane 412a, 412b is the tensioned penetrable zones 416a, 416b. Outside of the wire circles 408a, 408b, there is no material between the wires 404 of the wire frame 414. Thus fluid (e.g., blood) may travel along its native flow path within the atrium of the patient's heart through the device 400 without being materially obstructed by the device 400.

As a non-limiting example, device 400 can be pre-positioned within left atrium of the heart in order to conduct a transcatheter procedure via the chambers of the heart to implant a micropump in the left ventricle of the heart. In such a case, the device 400 would be transcatheterly implanted within left atrium of the heart with one of the tensioned penetrable zones 416a abutting the atrial septum and the other of the tensioned penetrable zones abutting the mitral valve. Such positioning would allow a catheter (e.g., a delivery sheath through which the procedure could be contacted) to enter the right atrium, pass through a breach in the atrial septum, penetrate through the first tensioned penetrable zone 416a, pass through the hollow interior of the sphere (formed by wire frame 414), penetrate through second penetrable zone 416b, through the mitral valve and into the left ventricle. In this example, in addition to its sealing effect, the device 400 has the effect of maintaining the catheter 60 properly in place as it passes from the right atrium through device 400 in the left atrium into the left ventricle. The device 400 supports the catheter 60 at its point of entry into the left atrium (at the atrial septum) and its point of exit from the left atrium (at the mitral valve).

For the sake of brevity, only the material differences between device 400 and the device 100 have been described. For the remainder of details, the reader is referred to the description of device 100 above, substituting the reference numbers 4xx for the reference numbers lxx in that description.

Referring to FIGS. 14, 15 and 16, there is shown another embodiment of the present technology, device 500, in its deployed configuration.

The device 500 has a support frame 502 and a cavity wall barrier membrane 512. In this embodiment the support frame 502 of the device 500 is a wire frame 514. The wire frame 514 is similar to an Amplazter occluder, but device 500 has a central lumen 536 through which a catheter 60 can pass, as is described in further detail below.

The wire frame 114 has an expanded configuration (in which it is shown in FIG. 14) and a collapsed configuration. As can be seen in FIG. 14 the wire frame 114, when in its expanded configuration, the wire frame 414 has a shape of two generally parallel spaced-apart discs 530a, 530b connected together at their central portions 532a by a hollow cylinder 534. The hollow cylinder forms the lumen 536 between the central portions 532a of the discs 530a, 530b, each of the discs 530a, 530b and the cylinder 534 having a radius, the radius (R_{C}) of the cylinder 534 being smaller than the radii (R_{D}) of each of the discs 530a, 530b. (In this embodiment the radii of the (R_{D}) discs 530a, 530b are equal. In other embodiments that is not be the case.)

The wire frame 514 is composed of a number of wires 504 that form one disc 530a, then the cylinder 534 and then the other disc 530b. As can be seen in FIG. 14, the wires 504 form a “flower-like” pattern on the discs 530a, 530b, and extend parallel to each other in the cylinder 534. This pattern ensures that the wire network 514 may be properly collapsed when in its compact configuration. As can also be seen in the FIG. 14, the wires 504 are connected together in multiple times in multiple places.

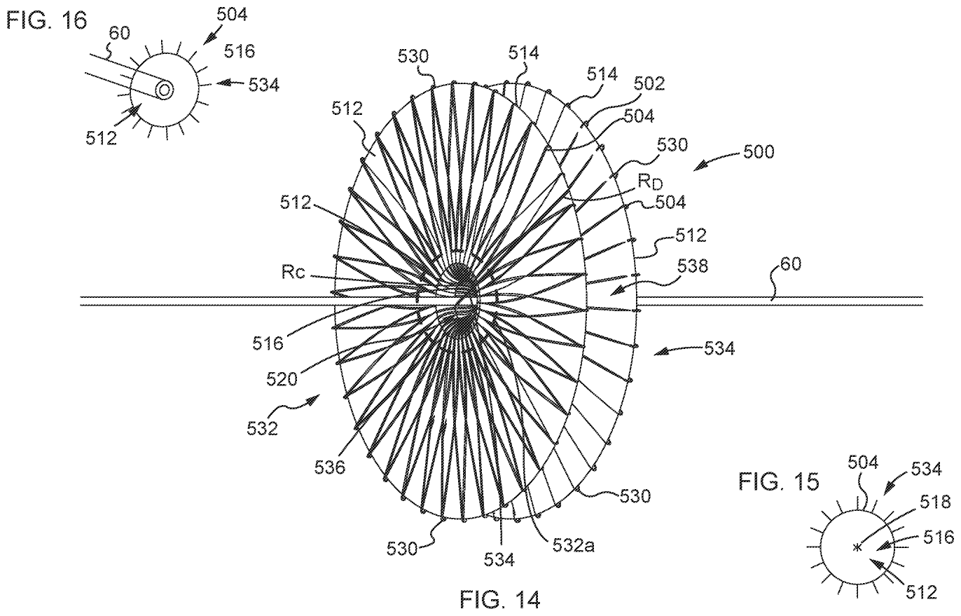

In this embodiment, as can been seen in FIG. 14 the barrier membrane 512 extends across the entire area of the each of the discs 530a, 530b (in the interior 538 of the device 500 with the barrier membrane 512 portions on each disc facing the other). The barrier membrane 512 extends around the periphery of the cylinder 534 as well. Finally, the barrier membrane 516 also extends across the lumen 536 formed in the cylinder 534 (see FIG. 15). As the wire frame 514 is in its expanded configuration, the barrier membrane 512 is under tension imparted by the wire frame 514. The portion of the barrier membrane 512 tensioned across the lumen 536 forms the tensioned penetrable zone 516 in this embodiment. The tensioned penetrable zone has one weakened zone 518 (FIG. 15).

The device 500 is useful when performing transcatheter procedures involving a transcaval technique (although it is not limited to such uses). In such a case, the device 500 is implanted such that in its deployed configuration one disc 530a is in its expanded configuration within the patient's vena cava, the other disc 530b is in its expanded configuration within the patient's aorta, and the cylinder 534 traverses a breach in the wall of the vena cava and a breach in the wall of the aorta. The disc 530a within the vena cava is positioned such that the barrier membrane 512 abuts the wall of the vena cava, which, in combination with the portion of the barrier membrane 512 extending around the periphery of the cylinder 534, prevents outflow of blood through the breach in the wall of the vena cava through which the cylinder 534 passes. Similarly, the disc 530b within the aorta is positioned such that the barrier membrane 512 abuts the wall of the aorta, which, in combination with the portion of the barrier membrane 512 extending around the periphery of the cylinder 534 preventing outflow of blood through the breach in the wall of the aorta through which the cylinder passes as well.

The tensioned penetrable zone 516 (extending across the lumen 536) prevents passage of blood from the aorta to the vena cava (and vice versa). During the course of the transcatheter procedure, a catheter 60 will penetrate the penetrable zone 516 (FIG. 16), and the material of the penetrable zone 516 will self-seal around the exterior of the catheter 60. Thus, the catheter is able access the aorta from the vena cava without blood outflowing from either blood vessel.

For the sake of brevity, only the material differences between device 500 and the device 100 have been described. For the remainder of details, the reader is referred to the description of device 100 above, substituting the reference numbers 5xx for the reference numbers lxx in that description.

### Miscellaneous

The present technology is not limited in its application to the details of construction and the arrangement of components set forth in the preceding description or illustrated in the drawings. The present technology is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the description the same numerical references refer to similar elements.

It must be noted that, as used in this specification and the appended claims, the singular form “a”, “an” and “the” include plural referents unless the context clearly dictates otherwise.

As used herein, the term “about” or “generally” or the like in the context of a given value or range (whether direct or indirect, e.g., “generally in line”, “generally aligned”, “generally parallel”, etc.) refers to a value or range that is within 20%, preferably within 10%, and more preferably within 5% of the given value or range.

As used herein, the term “and/or” is to be taken as specific disclosure of each of the two 10 specified features or components with or without the other. For example, “A and/or B” is to be taken as specific disclosure of each of (i) A, (ii) B and (iii) A and B, just as if each is set out individually herein.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1.-87. (canceled)

88. A medical implant for providing access to a body conduit system, the medical implant comprising:
a support frame defining a lumen and having an expanded configuration, the support frame in the expanded configuration being configured to anchor the medical implant in the body conduit system and to have the lumen in continuity with the body conduit system when the support frame is anchored in the body conduit system; and
a barrier membrane configured to couple to the support frame, the barrier membrane being tensioned by the support frame and penetrable for providing access to the lumen, when coupled to the support frame and when the support frame is in the expanded configuration.

89. The medical implant according to claim 88, further comprising an identifiable marker for assisting in positioning the medical implant in the body conduit system.

90. The medical implant according to claim 88, further comprising an identifiable marker for assisting in positioning the barrier membrane in the body conduit system to penetrate the barrier membrane.

91. The medical implant according to claim 88, wherein the support frame in the expanded configuration has a cylindrical shape defining the lumen longitudinally therethrough, the barrier membrane being circumferentially attached to the support frame.

92. The medical implant according to claim 88, wherein the support frame in the expanded configuration has a spherical shape, a spheroidal shape, an ellipsoidal shape, or a conidial shape, the lumen being centrally arranged relative to the support frame and defining two support frame openings, the barrier membrane being peripherally attached to the support frame and extending over at least one of the two support frame openings.

93. The medical implant according to claim 88, wherein the support frame in the expanded configuration has a shape comprising two spaced-apart discs connected together by a hollow cylinder defining the lumen longitudinally therethrough, the barrier membrane being attached to the support frame and extending across the lumen.

94. The medical implant according to claim 88, wherein the barrier membrane is configured to be fully contained within the body conduit system when the medical implant is implanted therein and the support frame is in the expanded configuration.

95. The medical implant according to claim 88, wherein the membrane barrier comprises a tensioned penetrable zone.

96. The medical implant according to claim 88, wherein the membrane barrier comprises a weakened site.

97. The medical implant according to claim 88, wherein the membrane barrier is made of a material comprising a hemostatic material.

98. The medical implant according to claim 88, wherein the membrane barrier is made of a material comprising a self-sealing material.

99. The medical implant according to claim 88, wherein the membrane barrier is made of a material comprising an elastomeric material.

100. The medical implant according to claim 88, wherein the membrane barrier is made of a material comprising a material having a Shore A Durometer of less than 90.

101. The medical implant according to claim 88, wherein the membrane barrier is made of a material comprising a silicone material.

102. The medical implant according to claim 88, wherein at least one of the support frame and the barrier membrane is made of a material comprising a resorbable material.

103. The medical implant according to claim 88, wherein the medical implant is sized and shaped to be implanted transcatheterly.

104. A method of providing access to a body conduit system, the method comprising:
implanting a medical implant in the body conduit system, the medical implant comprising a support frame defining a lumen that is in continuity with the body conduit system when the medical implant is implanted therein, and a barrier membrane coupled to the support frame;
obtaining an access opening to the body conduit system at an access site thereof that at least partially overlaps with the barrier membrane of the implanted medical implant; and
penetrating the barrier membrane to provide access to the lumen and the body conduit system.

105. The method according to claim 104, further comprising overlapping the barrier membrane of the implanted medical implant at least partially with the access site of the body conduit system based on an identifiable marker that is provided to the medical implant.

106. The method according to claim 104, wherein implanting the medical implant comprises obtaining a medical implant access opening to the body conduit system at a given site other than the access site.

107. The method according to claim 106, further comprising closing the medical implant access opening.

108. The method according to claim 104, wherein implanting the medical implant comprises anchoring the medical implant with the support frame in an expanded configuration in the body conduit system.

109. The method according to claim 104, wherein penetrating the barrier membrane comprises penetrating a tensioned barrier membrane.

110. The method according to claim 109, wherein the tensioned barrier membrane is tensioned by the support frame in an expanded configuration.

111. The method according to claim 104, wherein penetrating the barrier membrane comprises puncturing the barrier membrane.

112. The method according to claim 104, wherein obtaining the access opening to the body conduit system is performed before penetrating the barrier membrane.

113. The method according to claim 112, wherein with the barrier membrane penetrated an outflow of fluid from the body conduit system is at least partially prevented, compared to an outflow of fluid caused by obtaining an access opening to the body conduit system at the access site thereof in absence of the medical implant implanted in the body conduit system.

114. The method according to claim 104, wherein the medical implant is implanted in a blood vessel.

115. The method according to claim 104, wherein the medical implant is implanted in a chamber of the heart.

116. The method according to claim 104, wherein the medical implant is implanted in the inferior vena cava and the descending aorta.

117. The method according to claim 104, wherein the medical implant is implanted transcatheterly.
